(19) United States
(12) Patent Application Publication
(10) Pub. No.: US 2025/0177110 A1
(43) Pub. Date: Jun. 5, 2025

(54) FASCIAL CLOSURE DEVICE

(71) Applicant: Davol Inc., Warwick, RI (US)

(72) Inventor: Christian Fay, Scituate, RI (US)

(73) Assignee: Davol Inc., Warwick, RI (US)

(21) Appl. No.: 18/839,962

(22) PCT Filed: Feb. 2, 2023
(86) PCT No.: PCT/US2023/012181

§ 371 Date: Aug. 20, 2024

Related U.S. Application Data
(60) Provisional application No. 63/313,625, filed on Feb. 24, 2022.

(51) Int. Cl. A61F 2/00

(52) U.S. Cl. CPC A61F 2/0063; A61F 2220/0008; A61F 2220/0075

(57) ABSTRACT

An implantable prosthesis may include a body, at least one tether extending from a portion of the body, and at least one eyelet associated with the body. The prosthesis may be employed in a soft tissue repair procedure, serving to distribute stresses from nearby anatomy (e.g., intra-abdominal forces in a ventral hernia repair) to improve repair outcomes. In operation, the body may be positioned against a defect, the tether may be threaded through the muscle or tissue wall surrounding the defect, passed across the defect to an eyelet, and passed through the eyelet to secure the prosthesis. The tether may be attached to the surrounding tissue through any suitable fastener. The prosthesis may be used in minimally invasive surgeries such as laparoscopic and/or robotic surgeries.

### CROSS-REFERENCE TO RELATED APPLICATIONS

This application claims the benefit of U.S. Provisional Application No. 63/313,625, filed Feb. 24, 2022, which is hereby incorporated by reference in its entirety.

### FIELD

Disclosed embodiments are related to implantable mesh prostheses and related methods of use. More specifically, methods and apparatuses related to mesh prostheses with load distribution properties are disclosed.

### BACKGROUND

A hernia defect is an opening or weakness in a tissue or muscle wall, such as the abdominal wall. One approach for repairing a hernia is to cover the tissue wall defect with an implantable soft tissue prosthesis such as a patch of repair fabric or mesh.

One technique for repairing a soft tissue defect, such as an abdominal wall hernia, involves inserting the soft tissue prosthesis into an intra-abdominal space, positioning or aligning the prosthesis relative to the wall defect, and then, if desired, securing the prosthesis with tacks, sutures, and/or adhesives.

The prosthesis may also be delivered through a minimally invasive technique, such as a laparoscopic procedure. To deliver the prosthesis intra-abdominally, the prosthesis may be rolled up, folded or otherwise collapsed into a reduced configuration and then inserted through a small incision or a trocar and into the intra-abdominal space. The prosthesis is then unfurled and positioned relative to the defect.

### SUMMARY

In some aspects, implantable prostheses are provided. In some embodiments, the implantable prosthesis includes a body of biologically compatible repair fabric, the body having a periphery, at least one tether extending from a first portion of the periphery of the body, and at least one eyelet located associated with a second portion of the periphery of the body. The at least one eyelet is configured to receive the at least one tether therethrough. The at least one tether is configured to span across a portion of the body to pass through the at least one eyelet.

In some aspects, methods of tissue repair are provided. In some embodiments, the method of tissue repair includes act (a) delivering an implantable prosthesis to a tissue defect site on a tissue wall. The implantable prosthesis includes a body having a periphery, at least one tether extending from a first portion of the periphery, and at least one eyelet located proximate to a second portion of the periphery. The method also includes (b) positioning the body over the tissue defect on a first side of the tissue wall, (c) extending the at least one tether across the second side of the tissue wall located opposite the first side of the tissue wall, and (e) inserting the at least one tether through the at least one eyelet on the first side of the tissue wall to secure the body over the tissue defect.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 schematically illustrates an implantable prosthesis according to some embodiments;

FIG. 2 schematically illustrates a perspective view of an implantable prosthesis according to other embodiments;

FIG. 3 schematically illustrates an internal view of an implantable prosthesis positioned on soft tissue according to some embodiments;

FIG. 4 schematically illustrates an internal view of an implantable prosthesis positioned on soft tissue according to other embodiments; and

FIG. 5 schematically illustrates an external view of the implantable prosthesis from FIG. 4.

### DETAILED DESCRIPTION

In conventional laparoscopic soft tissue repair procedures, surgeons often place an implantable prosthesis in an internal biological space or pocket of a subject to provide support and/or strength to the defected tissue and expedite the repair process. For example, in a hernia repair procedure, an operator (e.g., surgeon and/or robotic tool) may place the prosthesis in the extraperitoneal plane. The prosthesis can be formed of a mesh-like sheet, slightly larger than the defect. The high surface area of the porous prosthesis can induce tissue ingrowth during the repair process. The prosthesis is typically sufficiently flexible to conform to the curved surfaces of the anatomical site of interest and move along with the subject, reducing the risk of discomfort.

The operator may deliver the prosthesis to an internal biological space or pocket with one or more surgical tools and/or robotic end effectors inserted into the surgical site. The prosthesis is then manipulated, positioned and/or oriented relative to the defect and subsequently secured with fasteners such as sutures, staples, tacks, barbs, hooks, etc. The forces necessary to close or otherwise repair certain defects (e.g., large ventral hernias greater than 6-10 cm) may result in failure of the fasteners. For example, sutures tied to the periphery of a prosthesis may pull-through, or tear through, surrounding tissue. Such failures, and in some cases, the introduction of new defects due to tears from the fasteners can result in complications and relapse.

The Inventors have recognized that many of these instances of suture pull through and other prosthesis failures can be attributed to uneven stress distribution and the inability of the operator to properly gauge and tension the fastener. With limited to no tactile feedback during robotic and/or laparoscopic procedures, the operator may unintentionally over-tension the fastener and/or in some cases, the prosthesis itself. This excess stress, in combination with the small footprint of many conventional fasteners, may result in a high point load in one or more locations of the soft tissue surrounding the defect.

Based on the foregoing, the Inventors have recognized the benefits associated with an implantable prosthesis which may distribute stresses from the defect closure site (e.g., intra-abdominal stresses) and communicate stresses along the prosthesis to the operator. The prosthesis may alleviate tension from the closure site to reduce the risk of failure and/or relapse. The implantable prosthesis may indicate in-line stresses from the prosthesis to the operator, allowing the operator to control the stresses placed on the prosthesis and/or fastener used to attach the prosthesis to the anatomy. In this way, the operator may more suitably tension the prosthesis for improved repair outcomes and a reduced risk of complications.

In some embodiments, an implantable prosthesis may include a main body and one or more tethers extending outwardly from the body. The tethers may be configured to affix the body at a defect site. The tethers may be sized to pass across the defect site to better distribute tension from forces in the closure site. For example, in embodiments where the prosthesis is used in a ventral hernia repair procedure, intra-abdominal forces may apply stresses to the prosthesis. As such, tethers may be configured with a size and/or shape to distribute the stresses along the intra-abdominal wall to reduce the risk of implant failure.

In some embodiments, the implantable prosthesis may include one or more eyelets associated with the body. In some embodiments, the one or more eyelets may correspond to the tethers. For example, in some embodiments, the eyelets may be positioned along portions of the body across from the tethers. The eyelets may be formed through an in-plane thickness of the body (e.g., from a first face to a second face), such that one or more tethers may be passed through the eyelets. The eyelets may serve as an anchoring feature to allow the tethers to pass across the defect at least once. In some embodiments, the tethers may be passed through the eyelets and subsequently pass back across the defect and/or another portion of the surrounding tissue, which may serve to close or approximate the defect. The tethers may then be secured to nearby tissue and/or a portion of the prosthesis to close or approximate the defect and maintain the defect in the closed or approximated configuration. Of course, in some embodiments, the tethers may be secured to the eyelets, tissue near the eyelets, and/or a portion of the body proximal to the eyelets, as the present disclosure is not limited by the location where the tethers are secured to close or approximate the defect. The tethers may be secured to any suitable location without passing back across the defect. The operator may elect to secure the tethers at any suitable position on the body and/or tissue depending on the defect size, location, and/or any other parameter of the procedure.

The tether and eyelet pairing may be arranged to function similarly to a tightening procedure of a belt and belt buckle, such that a body may be encircled by a belt (i.e., tethers) and tightened using the buckle loop (i.e., eyelets) as leverage to be tightened about the body.

In some embodiments, the eyelets may be arranged on tabs extending outwardly from a perimeter of the main body. The tabs may be sized smaller than the body, such that they may be more flexible, allowing the eyelets to be manipulated by the operator and/or tethers during use. In this way, the tabs may reduce the in-plane stresses applied to the main body, which may function as a structural support for the defect.

It should be appreciated that the implantable prostheses of the present disclosure may include any number of tethers and/or any number of eyelets, as the present disclosure is not so limited. In some embodiments, the number of tethers and eyelets may be different from one another to provide an operator with flexibility to decide how to proceed during the procedure. In other words, an implantable prosthesis may have excess eyelets and/or tethers to provide multiple alternative options of closure to the operator. In some embodiments, more than one tether may be threaded through a single eyelet.

In some embodiments, the tethers may be configured to be threaded through soft tissue surrounding the defect to secure the body. The tethers may have a cross-sectional aspect ratio sufficient to better distribute tension as compared to a relatively narrow suture. The tethers may have a width greater than their thickness (measured normal to the tissue plane), such that the tether remains flexible, but is capable of distributing forces (e.g., defect closure forces) across an area of tissue adjacent the defect, which may, in some embodiments, be a relatively large area. In other words, the tethers may be less likely to pull through or tear the anatomical structure due to their size and structure (e.g., mesh). To assist in penetration of tissue, in some embodiments, the tethers each may include a needle positioned at their distal end to facilitate passage of the tethers through the anatomy.

In some embodiments, the body and/or tethers may be formed of a flexible porous material including, but not limited to, a mesh. In its unstressed configuration, the body and/or tethers may have a first appearance associated with an average pore size of the porous material. Light may pass through the pores and render the material partially translucent. In a stressed configuration, or when the material undergoes strain in an in-plane direction, such as along an axial direction of the tethers, the pores of the material may collapse and stretch to accommodate the applied strain. Accordingly, less light may pass through the pores, and the material may have a second appearance. The operator may be able to use this variation in visual appearance (e.g., from a more translucent to a less translucent appearance) as an indicator of the strains and/or stresses within the porous material. In embodiments where the porous material has a negative Poisson's ratio, the change in cross-sectional area of the applied strain and/or stress may be visually apparent. For example, if the porous material is stretched along a first direction, its thickness along an orthogonal or otherwise related direction may be reduced relative to the applied strain. In this way, the operator may be able to determine the stresses along the material. The change in appearance relative to the strain in material may be particularly beneficial for the operator when tensioning or arranging the tethers relative to the defect. In some embodiments, a visual appearance of the tethers (and/or any other portion of the prosthesis body) may have at least a first configuration indicative of the tethers being not tensioned and at least a second configuration indicative of the tethers being tensioned, which may be distinct from the first configuration. In other words, the visual appearance of a tensioned porous material may be different from the visual appearance of an untensioned porous material.

In some embodiments, the prosthesis body may be planar, such that it may have a first face which may be positioned against a tissue or muscle wall (e.g., an abdominal wall) including the defect, as well as a second face, which may be located adjacent to sensitive nearby organs (e.g., intestines and/or other viscera). As such, the first face, which may be referred to in some embodiments as the anterior face, may be configured for tissue ingrowth. In some embodiments, the anterior face may be rough and porous, with an increased surface area which may induce tissue ingrowth during a soft tissue repair procedure (e.g., hernia repair)-expediting the repair process. The second face, which may be referred to in some embodiments as the visceral face, may be configured to reduce the risk of adhesions forming with the visceral organs. As such, the visceral face may be smoother and/or less porous (e.g., microporous) than the anterior face, and may include one or more anti-adhesion barrier layers.

In a minimally invasive technique, as well as in certain open procedures, an implantable prosthesis may be reduced in size to fit a defect and/or facilitate delivery of the prosthesis to the surgical site. For example, in a laparoscopic procedure, the implantable prosthesis may be rolled into a slender cylindrical shape, or otherwise collapsed into a smaller configuration, suitable for passage through a narrow cannula which may have an inner diameter of approximately 10 mm, of approximately 5 mm, or even a smaller size. The prosthesis may then be unfurled and directed to a defect site within the surgical pocket with one or more tools. The prosthesis may subsequently be fixed against the soft tissue repair site with any suitable fasteners (e.g., barbs, sutures, tacks, staples, hooks, etc.). Of course, the implantable prostheses described herein may be employed in any conventional soft tissue repair procedures.

It should be appreciated that any portion of the prosthesis body may be formed of any suitable biocompatible material and/or combinations of materials. In some embodiments, the prosthesis may be formed of a knitted polypropylene mesh material. The tabs and/or tethers may either be formed of the same or different biocompatible material as the prosthesis body. For example, the tethers may be formed of a biocompatible material with greater in-plane flexural strength than a biocompatible material used in the prosthesis body with specific repair properties. As such, the tethers may be attached or otherwise affixed to the body after formation. In some embodiments, the implantable prosthesis may include one or more coatings (e.g., to prevent adhesion with visceral organs, to enhance tissue cell growth, to deliver pharmaceutical materials, etc.) on one or more portions (e.g., body, a portion and/or face of the body, tethers, a portion and/or face of the tethers, tab, a portion and/or face of the tabs). For example, in some embodiments, the tethers of the prosthesis may include an antibiotic or antimicrobial coating to prevent wound dehiscence and other related complications. In some embodiments, the entire prosthesis may be coating with a biocompatible material on at least one face.

In some embodiments, the implantable prosthesis may be formed from a stock sheet of mesh material, such that the entire implantable prosthesis, including the body, the tethers and the tabs, may be formed as a unitary or monolithic structure of the same material. In other embodiments, at least one iteration of at least one feature (e.g., tabs, eyelets, tethers) may be formed of another material, and may be attached to the prosthesis through any suitable technique, such as with heat, ultrasonic, induction, vibration, infrared, laser, and/or any treatment. For example, the tethers may be formed from a different mesh and/or material than the body, and subsequently attached to (e.g., via welding or embroidery techniques) the body. An operator may elect to have a non-monolithic implantable prosthesis which may exhibit non-uniform mechanical properties (e.g., better flexibility in the tethers, better strength in the body) for more optimized implant placement and wound closure.

It should be appreciated that any suitable biocompatible material or combination of materials may be employed to form any portion of the implantable prostheses, as the present disclosure is not limited by the material composition or arrangement of the prostheses.

In some embodiments, the implantable prosthesis may be formed from a stock sheet of fabric and/or mesh material (e.g., polypropylene mesh) by die cutting, laser cutting, water jets, hand-cutting, and/or any other suitable technique. In some embodiments, the size and/or shape of the implantable prosthesis may be selected to match a corresponding size and/or shape of a defect. As such, an operator may measure the defect and/or features of the surgical pocket and subsequently cut down an initial first shape and/or size of the implantable prosthesis (e.g., with blades or scissors) to better customize the prosthesis to the defect site. Accordingly, the present disclosure is not limited by the size and/or shape of the prosthesis or any portion of the prosthesis (e.g., tabs, eyelets, tethers).

The term “biocompatible materials” used herein refers to materials that have the ability to perform with an appropriate host response in a specific application. Biocompatible materials have the quality of not having toxic or injurious effects on biological systems. In some embodiments, biocompatible materials may be biologically compatible.

As used herein, the terms “resorbable” or “biodegradable” refer to materials that are degraded by the body's enzymatic and/or hydrolytic pathways through a reaction against “foreign” material. Depending on the chemical nature of the material, the bioabsorbable material may disappear into the in vivo environment after a defined period, which can vary, for example, from a few hours to several months.

Examples of resorbable materials include, but are not limited by, polylactic acid (PLA), polyglycolic acid (PGA), oxidized cellulose, polycaprolactone (PCL), polydioxanone (PDO), trimethylene carbonate (TMC), polyvinyl alcohol (PVA), polyhydroxyalkanoates (PHAs), polyamides, polyethers, copolymers thereof, and/or and mixtures thereof.

Examples of non-resorbable materials include, but are not limited by, polyethylene terephthalate (PET), polyamides, aramids, expanded polytetrafluoroethylene, polyurethane, polyvinylidene difluoride (PVDF), polybutyl esters, polyetheretherketone (PEEK), polyolefins (such as polyethylene or polypropylene), copper alloys, silver alloys, platinum, medical grades of steel such as medical-grade stainless steel, and/or combinations thereof.

The term “implantable prosthesis” used herein refers to a non-limiting manner to a flexible plane member (e.g., a mesh or patch) of desired contour, selected in a non-limiting manner from biocompatible compositions selected from polymeric compositions; glassware; titanium containing, stainless steel, nitinol (Nickel Titanium alloys), and/or other metal ware; composite materials; cardboard, natural fiber, silicone, rubber or rubber-like compositions or any mixture thereof. In some embodiments, the implantable prosthesis may be formed of polypropylene.

The term “minimally invasive surgery” used herein refers to a procedure that is carried out by entering the body through the skin or through a body cavity or anatomical opening, but with the smallest damage possible.

The term “trocar” used herein refers to a surgical instrument passed through the body or abdominal wall, used to allow easy exchange of endoscopic instruments during endoscopic or other minimally invasive surgery.

The term “hernia” used herein refers to a hernia in the abdominal cavity or in pre-peritoneal. Moreover, the term hernia may be regarded as umbilical hernia, hiatal hernia, ventral hernia, postoperative hernia, epigastric hernia, spiegelian hernia, inguinal hernia and femoral hernia, generally any abdominal wall related hernia.

It should be appreciated that the implantable prosthesis of the present disclosure may be employed in any suitable repair application, including, but not limited to, hernia repair, pelvic mesh, breast implant support, repair patch for the dura mater, inguinal hernia repair, combinations thereof, and/or any other suitable application. In some embodiments, the implantable prosthesis may be used in a repair process for a defect formed in a soft tissue.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

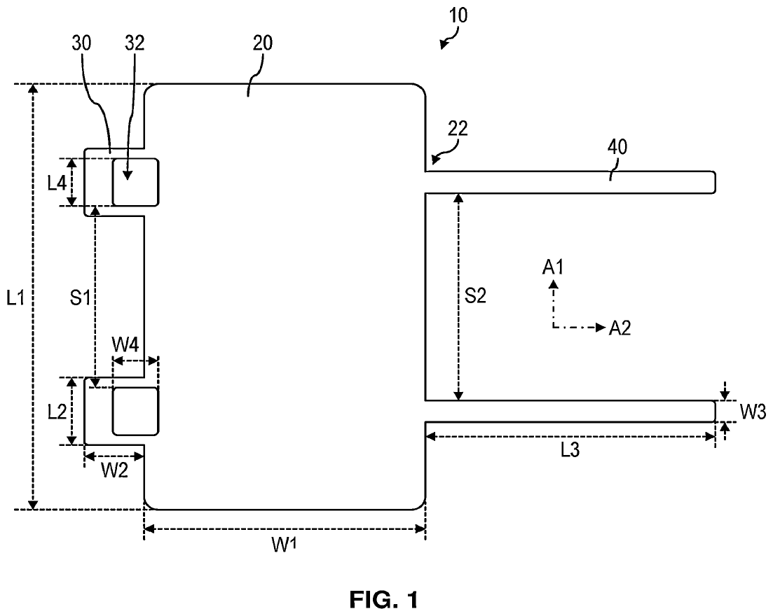

FIG. 1 shows an implantable prosthesis 10 according to some embodiments. The prosthesis 10 may include a main body 20, with one or more tabs 30 and one or more tethers 40. Each tab 30 may include at least one eyelet 32, configured to accept at least one tether 40. The prosthesis may be formed of a flexible material (e.g., polypropylene mesh), such that the body 20 may be conformable against complex anatomical surfaces of interest.

It should be appreciated that the main body may be any suitable shape to fit within the soft tissue pocket of an anatomical defect. The main body may be cut or otherwise geometrically adjusted to better fit a given defect. In some embodiments, the main body's geometric parameters (e.g., width, length, perimeter, etc.) may be determined by the defect size. For example, an operator may first inspect a soft tissue defect, measure critical geometric parameters, and subsequently adjust the size of the main body to fit the defect size. The body size may therefore be determined by a multiple of a measured effective length and width of the defect in some embodiments. In some embodiments, the main body may be planar or sheet-like, such that it may be flexible and conformable about anatomical features. In other embodiments, the main body may be non-planar, such that it may be curved or three-dimensional. In some embodiments, the body may be pre-formed with a non-planar configuration. In other embodiments still, the main body may include one or more planar portions, and one or more non-planar portions. Accordingly, the implantable prostheses of the present disclosure are not limited by the geometry or shape of the main body.

In some embodiments, the main body 20 may be defined by at least two dimensions. For example, as shown in FIG. 1, the main body 20 may be defined by a body length L1 along a first direction A1 and a body width W1 along a second direction A2. In some embodiments, at least one of the body length L1 and body width W1 may be greater than or equal to 1 cm, 2 cm, 3 cm, 5 cm, 7 cm, 10 cm. 12 cm, 14 cm, 20 cm, and/or any other suitable size. At least one of the body length L1 and body width W1 may also be less than or equal to 20 cm, 14 cm, 12 cm, 10 cm, 7 cm, 5 cm, 3 cm, 2 cm, 1 cm, and/or any other suitable size. Combinations of the foregoing ranges are also contemplated, including, for example, one or both of the body length L1 and body width W1 being between 1 cm and 20 cm, 2 cm and 10 cm, and/or any other suitable range. Of course, other ranges, including ranges both greater than and less than those noted above are also contemplated as the present disclosure is not so limited.

It should be appreciated that the body width W1 and body length L1 may be selected to accommodate a given defect size. In other words, a defect size in combination with a given overhang or tolerance may help determine the body length L1 and width W1. The overhang may serve as a buffer or allowance around the defect to reduce the risk of the implantable prosthesis from uncovering the wound. In some embodiments, the overhang may provide extra strength to the implantable prosthesis, which may be under tension during wound closure. For example, for a wound with an effective width of approximately 2 cm and an effective length of approximately 8 cm, an implantable prosthesis with a body width of 8 cm (corresponding to the wound width and an overhang of 4 cm, 2 cm on each side of the wound) and a body length of 14 cm (corresponding to the wound length and an overhang of 4 cm. 2 cm on each side of the wound) may be employed. It should be appreciated that any suitable overhang size may be employed, including, but not limited to, greater than or equal to 1 cm, 2 cm, 3 cm, 4 cm, 5 cm, 6 cm, 8 cm, 10 cm, and/or any other suitable size. Overhangs less than or equal to 10 cm, 8 cm, 6 cm, 5 cm, 4 cm, 3 cm, 2 cm, 1 cm, and/or any other suitable size may also be employed, as the present disclosure is not so limited. Combinations of the foregoing ranges are also contemplated, including, for example, an overhang between 1 and 10 cm, 2 and 8 cm, and/or any other suitable range. Of course, other ranges, including ranges both greater than and less than those noted above are also contemplated as the present disclosure is not so limited.

It should be appreciated that in some embodiments, a wound may be centered about the prosthesis body, such that the overhang may be equal on both sides, whereas in other embodiments, an operator (e.g., surgeon) may elect to place the prosthesis off-center. It should also be appreciated that the overhang in the width direction may or may not be equivalent to the overhang in the length direction. The operator may not require extensive overhangs in one or more directions, and may elect to choose a smaller prosthesis instead.

It should be appreciated that the prosthesis 10 may have an in-plane thickness of any suitable size. In some embodiments, the in-plane thickness of the prosthesis may render the prosthesis flexible, such that it may be manipulatable and conformable against the soft topography of the biological pocket. In some embodiments, one or more portions (e.g., prosthesis body 20, tabs 30, and/or tethers 40) may have a different in-plane thickness than one or more other portions of the prostheses. It should be appreciated that the in-plane thickness may be any suitable thickness known in the art, as the present disclosure is not limited by the in-plane thickness of any portion of the implantable prostheses.

The prosthesis 10 may include one or more tabs 30 extending outwardly from the body 20. In some embodiments, one or more tabs 30 may be positioned on one side of the body 20, as shown in FIG. 1, whereas in other embodiments, one or more tabs may be arranged at various positions about the body. In some embodiments, the tabs 30, including the eyelets 32, may be positioned generally along the same axis as the tethers. For example, as shown in FIG. 1, two tabs may extend outwardly along the same direction A2 as two tethers 40. As described previously, the tethers may be configured to span across the body 20 during operation and pass through at least one eyelet 32. As such, it may be beneficial for the cyclets to be positioned approximately along the same direction as the tethers. However, in some embodiments, the eyelets and tethers may be arranged along different directions. The tethers may be sufficiently flexible to span across at least a portion of the body and reach an cyelet positioned anywhere on the prosthesis. As such, the present disclosure is not limited by the arrangement or distribution of the tabs and eyelets with respect to the tethers.

In some embodiments, the tabs 30 may be defined by at least two dimensions. For example, as shown in FIG. 1, the tabs 30 may have a tab length L2 measured in direction A1 and a tab width W2 measured in direction A2. The tabs may be sized to sufficiently support one or more eyelets 32, which may be configured to accommodate a tether 40 when securing the prosthesis 10. The tabs may have any suitable size relative to the body. In some embodiments, at least one of the tab length L2 and tab width W2 may be greater than or equal to 2%, 5%, 7%, 10%, 12%, 15%, 20%, 25%, 30%, 40%, 50%, 60%, 75%, 90%, 100%, and/or any other suitable percentage of the body length L1 or body width W1. At least one of the tab length L2 and tab width W2 may also be less than or equal to 100%, 90%, 75%, 60%, 50%, 40%, 30%, 25%, 20%, 15%, 12%, 10%, 7%, 5%, 2%, and/or any other suitable percentage of the body length L1 or body width W1. Combinations of the foregoing ranges are also contemplated, including, for example, one or both of the tab length L2 and tab width W2 between 2% and 100%, 5% and 50%, and/or any other suitable range of percentages of the body length L1 or body width W1. Of course, other ranges, including ranges both greater than and less than those noted above are also contemplated as the present disclosure is not so limited. It should be appreciated that although both tabs 30 are shown to have the same tab length L2 and tab width W2 in FIG. 1, embodiments of implantable prostheses with tabs with various lengths and/or widths are also contemplated, as the present disclosure is not limited by the length or variations in length of the tabs.

In some embodiments, as shown in FIG. 1, the prosthesis 10 may include tethers 40 extending outwardly from a main body 20. The tethers may more effectively distribute stresses from the defect closure site compared to other point-loaded fixation devices, such as sutures or tacks. The tethers 40 may extend a tether length L3 relative to the body width W1, measured along a direction A2. In some embodiments, the tether length L3 may be sufficiently long to allow the tethers to traverse a defect at least once. As will be described in greater detail below, the body 20 may be secured to the defect by passing the tethers 40 across the defect once, through at least one eyelet, and subsequently across at least a portion of the defect. As such, in some embodiments, the tether length L3 may be greater than a body width W1. However, in some embodiments, the tethers 40 may be arranged to pass across a smaller portion of the defect, such that the tether length L3 may not be greater than the body width W1. In addition, as described previously, in some embodiments, the tethers may visually indicate a level of in-line tension to an operator. As such, the tethers may be sufficiently long to be visible to the operator on one or more sides of a defect site.

In some embodiments, the tether length L3 may be greater than or equal to 4 cm, 5 cm, 6 cm, 8 cm, 10 cm, 12 cm, 15 cm, 16 cm, 18 cm, 20 cm, 25 cm, 30 cm, and/or any other suitable length. The tether length L3 may also be less than or equal to 30 cm, 25 cm, 20 cm, 18 cm, 16 cm, 15 cm, 12 cm, 10 cm, 8 cm, 6 cm, 5 cm, 4 cm, and/or any other suitable length. Combinations of the foregoing ranges are also contemplated, including, for example, tether lengths L3 between 4 cm and 30 cm, 10 cm and 16 cm, and/or any other suitable range of lengths. Of course, other ranges, including ranges both greater than and less than those noted above are also contemplated as the present disclosure is not so limited.

The tether length L3 may be any suitable size relative to the body width W1. In some embodiments, the tether length L3 may be greater than or equal to 20%, 30%, 50%, 75%, 100%, 125%, 150%, 175%, 200%, 225%, 250%, 275%, 300%, 400%, 500%, 600% and/or any other suitable percentage of the body width W1. The tether length L3 may also be less than or equal to 600%, 500%, 400%, 300%, 275%, 250%, 225%, 200%, 175%, 150%, 125%, 100%, 75%, 50%, 30%, 20%, and/or any other suitable percentage of the body width W1. Combinations of the foregoing ranges are also contemplated, including, for example, tethers with length L3 between 20% and 600%, 10% and 500%, and/or any other suitable range of percentages of the body width W1. Of course, other ranges, including ranges both greater than and less than those noted above are also contemplated as the present disclosure is not so limited.

It should be appreciated that although both tethers 40 are shown to have the same tether length L3 in FIG. 1, embodiments of implantable prostheses with tethers with various lengths are also contemplated, as the present disclosure is not limited by the length or variations in length of the tethers.

In some embodiments, the tethers may have a tether width W3, measured in a direction A1. The tether width W3 may form part of a cross-section (along with an in-plane thickness of the tethers) on which stresses from the closure procedure may be applied. As such, the tether width W3 may be selected to achieve proper distribution of stress in the prosthesis 10.

In some embodiments, the tether width W3 may be greater than or equal to 0.1 cm, 0.2 cm, 0.5 cm, 1 cm, 2 cm, 3 cm, 4 cm, 5 cm, 6 cm, 8 cm, 10 cm, and/or any other suitable width. The tether width W3 may also be less than or equal to 10 cm, 8 cm, 6 cm, 5 cm, 4 cm, 3 cm, 2 cm, 1 cm, 0.5 cm, 0.2 cm, 0.1 cm, and/or any other suitable width. Combinations of the foregoing ranges are also contemplated, including, for example, tether widths W3 between 0.1 cm and 10 cm, 2 cm and 4 cm, and/or any other suitable range of widths. Of course, other ranges, including ranges both greater than and less than those noted above are also contemplated as the present disclosure is not so limited.

In some embodiments, the tether width W3 may be determined by the body length L1. The tether width W3 may be any suitable size relative to the body length L1. In some embodiments, the tether width W3 may be greater than or equal to 2%, 5%, 7%, 10%, 12%, 15%, 20%, 25%, 30%, 40%, 50%, and/or any other suitable percentage of the body length L1. The tether width W3 may also be less than or equal to 50%, 40%, 30%, 25%, 20%, 15%, 12%, 10%, 7%, 5%, 2%, and/or any other suitable percentage of the body length L1 of the body. Combinations of the foregoing ranges are also contemplated, including, for example, tethers with widths W3 between 2% and 50%, 2% and 30%, and/or any other suitable range of percentages of the body length L1 of the body. Of course, other ranges, including ranges both greater than and less than those noted above are also contemplated as the present disclosure is not so limited. It should be appreciated that although both tethers 40 are shown to have the same tether width W3 in FIG. 1, embodiments of implantable prostheses with tethers with various widths are also contemplated, as the present disclosure is not limited by the width or variations in width of the tethers.

In some embodiments, the one or more tethers 40 may have an in-plane thickness measured normal to the defect plane. In embodiments where the body 20 and tethers 40 are formed from the same sheet of mesh, for example, the tethers 40 may have an in-plane thickness equivalent to the in-plane thickness of the body 20. Of course, embodiments in which the tethers 40 may have an in-plane thickness greater than or equal to the in-plane thickness of the body 20 are also contemplated.

In some embodiments, the tether width W3, as shown in FIG. 1, may be greater than the in-plane thickness of the tethers 40. The tether width W3 may be at least 105%, 110%, 120%, 130%, 150%, 175%, 200%, 300%, 400%, 500%, 600%, 700%, 800%, 900%, 1000%, 1500%, 2000%, 3000%, and/or any other suitable percentage of the in-plane thickness of the tethers 40. The tether width W3 may also be less than or equal to 3000%, 2000%, 1500%, 1000%, 900%, 800%, 700%, 600%, 500%, 400%, 300%, 200%, 175%, 150%, 130%, 120%, 110%, 105%, and/or any other suitable percentage of the in-plane thickness of the tethers 40. Combinations of the foregoing ranges are also contemplated, including, for example, a tether width W3 between 105% and 3000% or 200% and 2000% and/or any other suitable range of percentages of an in-plane thickness of the tethers 40. Of course, other ranges, including ranges both greater than and less than those noted above are also contemplated as the present disclosure is not so limited.

In some embodiments, an implantable prosthesis may include one or more eyelets formed in its main body, eliminating the need for tabs. The arrangement of eyelets directly in the main body may enhance the flexural strength of the eyelets, which may, in some embodiments, improve the capability of the implantable prosthesis to distribute stresses from the defect closure site. The eyelets may be positioned in close proximity to the perimeter of the prosthesis, such that that the tethers may be offset from the eyelets by at least a portion of the body. In some embodiments, the distance between the eyelets (whether in distinct tabs or arranged in the prosthesis body) may be related to a defect size.

The eyelets may be any suitable shape and/or size to accommodate the tethers. In some embodiments, the eyelets 32 may be defined by at least two dimensions along two directions of the prosthesis. For example, the eyelets 32 may have an eyelet width W4 measured in direction A2 and an eyelet length L4 in direction A1. In some embodiments, the eyelets 32 may be provided partially in the tabs 30 and partially in the body 20, as shown in FIG. 1. In other embodiments, the eyelets 32 may be provided only in the tabs and/or only in the body 20. As described previously, the tethers of the implantable prosthesis may be passed through the eyelets 32 before passing across the defect and/or the body 20. As such, the eyelet width W4 of the eyelets may be sized to accommodate an in-plane thickness of the tethers 40 of FIG. 1, and the eyelet length L4 of the eyelets may be sized to accommodate the tether width W3.

In some embodiments, the eyelet width W4 may be greater than or equal to 0.1 cm, 0.25 cm, 0.5 cm, 0.75 cm, 1 cm, 1.25 cm, 1.5 cm, 2 cm, 5 cm, and/or any other suitable width. The eyelet width W4 may also be less than or equal to 5 cm, 2 cm, 1.5 cm, 1.25 cm, 1 cm, 0.75 cm, 0.5 cm, 0.25 cm, 0.1 cm, and/or any other suitable width. Combinations of the foregoing ranges are also contemplated, including, for example, eyelet widths W4 between 0.1 and 5 cm, 0.5 and 2 cm, and/or any other suitable range of widths. Of course, other ranges, including ranges both greater than and less than those noted above are also contemplated as the present disclosure is not so limited. In some embodiments, the eyelet may be formed as a slit rather than an eyelet, such that the eyelet width W4 may be minimal. For example, if a laser cutting system is used to form the slit-like eyelets of the prosthesis, the eyelet width may be approximately equal to the laser beam width.

In some embodiments, the in-plane thickness of the tethers 40 may be at least 10%, 25%, 50%, 75%, 90%, 99%, and/or any other suitable percentage of the eyelet width W4. The in-plane thickness of the tethers 40 may also be less than or equal to 99%, 90%, 75%, 50%, 25%, 10%, and/or any other suitable percentage of the eyelet width W4. Combinations of the foregoing ranges are also contemplated, including, for example, an in-plane thickness of the tethers 40 between 10% and 99%, 10% and 90%, and/or any other suitable range of percentages of an eyelet width W4. Of course, other ranges, including ranges both greater than and less than those noted above are also contemplated as the present disclosure is not so limited.

In some embodiments, the eyelet length L4 may be commensurate in size, slightly larger than, and/or slightly smaller than the tether width W3. For example, in some embodiments, the eyelet length L4 may be 1 cm, and the tether width W3 may be 0.5 cm. In another example, the eyelet length L4 and tether width W3 may both be 1 cm, but the tether may be sufficiently flexible to fit into the eyelet to secure the prosthesis. In some embodiments, the eyelet length L4 may be greater than or equal to 0.1 cm, 0.2 cm, 0.5 cm, 1 cm, 2 cm, 3 cm, 4 cm, 5 cm, 6 cm, 8 cm, 10 cm, and/or any other suitable width. The eyelet length L4 may also be less than or equal to 10 cm, 8 cm, 6 cm, 5 cm, 4 cm, 3 cm, 2 cm, 1 cm, 0.5 cm, 0.2 cm, 0.1 cm, and/or any other suitable width. Combinations of the foregoing ranges are also contemplated, including, for example, eyelet lengths L4 between 0.1 cm and 10 cm, 2 cm and 4 cm, and/or any other suitable range of lengths. Of course, other ranges, including ranges both greater than and less than those noted above are also contemplated as the present disclosure is not so limited.

In some embodiments, the eyelet length L4 may be at least 100%, 110%, 125%, 150%, 175%, 200%, 300% and/or any other suitable percentage of the tether width W3. The eyelet length L4 may also be less than or equal to 300%, 200%, 175%, 150%, 125%, 110%, 100%, and/or any other suitable percentage of the tether width W3. Combinations of the foregoing ranges are also contemplated, including, for example, an eyelet length L4 between 100% and 300%, 125% and 200%, and/or any other suitable range of percentages of the tether width W3. Of course, other ranges, including ranges both greater than and less than those noted above are also contemplated as the present disclosure is not so limited.

It should be appreciated that the eyelet may be optically apparent or obvious to the operator, through either visual inspection of the surgeon and/or with surgical tools. As such, in some embodiments, the eyelet may be sized larger than an average pore size of the underlying prosthesis body 20. In this way, the operator may distinguish the eyelet over the body when attempting to thread one or more tethers through the eyelet. In some embodiments, the prosthesis may include one or more indicia (e.g., embroidery, colored markings, labels, arrows, etc.) which may direct the operator to one or more eyelets. It should be appreciated that the prosthesis may include any suitable marking or indicia known in the art to distinguish the eyelets (and/or any other feature of the prosthesis), as the present disclosure is not so limited. In some embodiments, the size and/or geometry of the eyelets relative to the surrounding prosthesis body material may sufficiently distinguish the eyelets from the body.

As shown in FIG. 1, in some embodiments, an implantable prosthesis 10 may include eyelets 32 spaced apart by an eyelet spacing S1 and tethers 40 spaced apart by a tether spacing S2. As noted previously, the eyelets and tethers (and, in some embodiments, tabs) of the present disclosure may be arranged in any suitable manner. The spacings S1, S2 may be determined by the number of eyelets and tethers, the eyelet length L4, tether length L3, as well as the body length L1. Accordingly, the present disclosure is not limited by the spacing between eyelets and/or tethers. It should be appreciated that although eyelets 32 and tethers 40 are shown to be on different sides of the body 20 in FIG. 1, embodiments in which eyelets and tethers are arranged on the same side of the body (with corresponding tethers and eyelets on the opposing side) are also contemplated.

It should be appreciated that although FIG. 1 illustrates an implantable prosthesis 10 with a pair of tabs 30 including eyelets 32 and a pair of tethers, the implantable prostheses of the present disclosure may include any suitable number of tabs, eyelets, and tethers. In some embodiments, the implantable prosthesis may include at least one, two, three, four, five, six, seven, eight, nine, ten, and/or any other suitable number of tabs, eyelets and/or tethers. The implantable prosthesis may also include less than or equal to ten, nine, eight, seven, six, five, four, three, two one, and/or any other suitable number of tabs, eyelets, and/or tethers. Combinations of the foregoing ranges are also contemplated, including, for example, the implantable prosthesis including between one and ten, one and five, and/or any other suitable range of numbers of tabs, eyelets, and/or tethers. Of course, other ranges, including ranges both greater than and less than those noted above are also contemplated as the present disclosure is not so limited. In some embodiments, the implantable prosthesis may include the same number of tabs, eyelets, and tethers. In other embodiments, the implantable prosthesis may include a different number of tabs, eyelets, and/or tethers relative to each other. As such, it should be appreciated that the implantable prostheses of the present disclosure are not limited by the number or arrangement of tabs, eyelets, and tethers.

It should be appreciated that although the tabs, eyelets, and tethers are all shown to be substantially rectangular with rounded edges in FIG. 1, the implantable prostheses of the present disclosure are not limited by the shape or arrangement of the tabs, eyelets, and tethers. Accordingly, any of the tabs, eyelets, and tethers may be any suitable shape (e.g., semi-circular, partially oval-shaped, polygonal, triangular, square-shaped, etc.) arranged at any suitable position relative to the body 20. In embodiments with more than one tab, eyelet, and/or tether, each tab, eyelet, and/or tether may be any suitable shape, irrespective of other similar features. It should be appreciated that the shapes, sizes, arrangement, distribution, and/or any other parameter of the tabs, eyelets and/or tethers may be selected to best support a given defect. In some embodiments, various corners (see, for example corner 22 in FIG. 1) may be rounded, chamfered, and/or reinforced (e.g., with more mesh, various coatings, embroidery, etc.) to reduce localized stresses in the body. The various parameters may also be selected to best suit the manufacturing method used to form the prosthesis. As such, the present disclosure is not limited by the shapes, sizes, arrangement, distribution, and/or any other parameter of the tabs, eyelets and/or tethers.

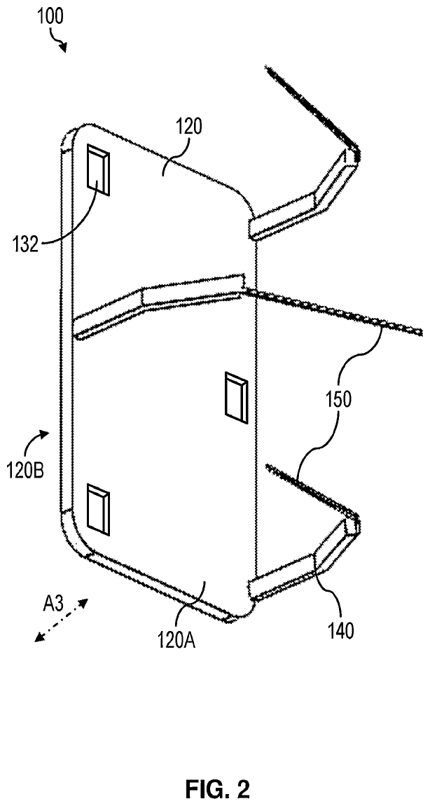

FIG. 2 shows an implantable prosthesis 100 according to some embodiments. The prosthesis 100 may include one or more tethers 140, which may extend from a body 120. It should be appreciated that although a planar body 120 is shown in FIG. 2, non-planar structures of the prosthesis body are also contemplated. The tethers 140 may be flexible, such that they may pass across a defect in a closure procedure. In some embodiments, the tethers may be configured to be threaded through soft tissue to secure the body 120. As shown in FIG. 2, in some embodiments, each tether 140 may include a needle 150 extending from a distal end of the tether, which may allow the tether 140 to penetrate soft tissue. The needle 150 may be any suitable needle used in soft tissue repair procedures, formed of any suitable biocompatible material, and in some embodiments, may be removed after the procedure. In some embodiments, the needle 150 may be pre-formed with the tether 140, whereas in other embodiments, the tether may be threaded with the needle 150 prior to use. It should be appreciated that the present disclosure is not limited by the structure, arrangement, attachment, and/or presence of the needles 150.

The prosthesis 100 of FIG. 2 may also include one or more eyelets 132 formed in the main body 120. As shown in the figure, in some embodiments, the eyelets 132 may be formed near a perimeter or periphery of the main body 120. Of course, embodiments in which the eyelets are positioned more centrally on the main body are also contemplated, as the present disclosure is not so limited. The eyelets 132 may extend from a first face 120A of the body 120 to a second face 120B. In other words, the eyelets 132 may extend through the total in-plane thickness of the body 120 measured along a direction A3. As will be described in greater detail below, the tethers 140 may be passed through the eyelets 132 in order to secure the prosthesis 100 to a defect.

As shown in FIG. 2, the eyelets 132 may be generally positioned on the body 120 across from the tethers 140. However, as noted previously, the eyelets may be arranged in any suitable position relative to the tethers, as the present disclosure is not so limited. Furthermore, although FIG. 2 shows a prosthesis 100 with tethers 140 and eyelets 132 arranged on both sides of the prosthesis body 120, it should be appreciated that the prostheses of the present disclosure may include eyelets and tethers arranged in any suitable manner with respect to one another (e.g., all tethers on one side, as shown in FIG. 1, tethers and eyelets on the same side, as shown in FIG. 2, etc.).

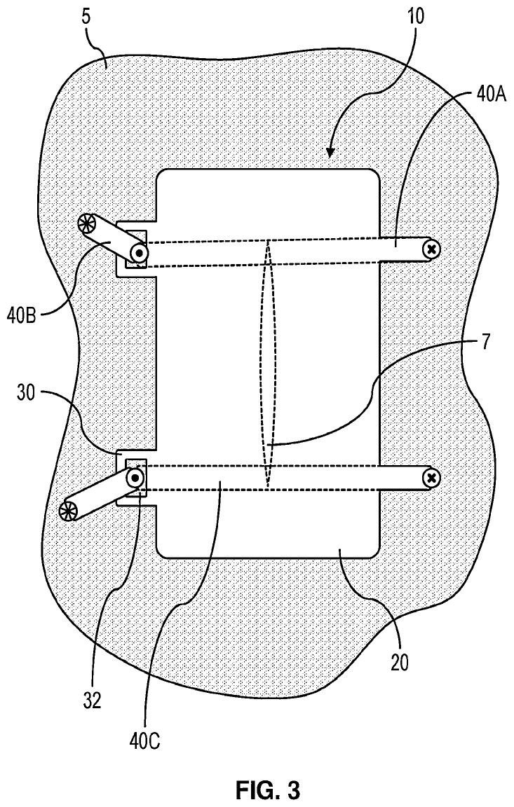

FIG. 3 depicts an implantable prosthesis 10 secured to a tissue or muscle wall 5. The prosthesis may be used in a defect closure procedure for a defect 7 formed in the tissue wall 5. FIG. 3 shows an internal view of an implantable prosthesis 10 including tabs 30 and tethers 40. The prosthesis 10 includes eyelets 32 formed in the tabs 30 to receive the tethers 40.

In operation, a prosthesis may be delivered to a surgical site or biological pocket and subsequently manipulated and oriented to cover the defect 7. As shown in FIG. 3, the prosthesis body 20 may be sized to be larger than the defect 7 to provide sufficient support during closure and healing. Once the body 20 has been suitably positioned over the defect 7, the tethers may be passed through the tissue wall 5 such that a proximal portion 40A of the tethers, proximal to the body 20, may extend through the tissue wall 5, represented by the in-plane (x) and a distal portion 40B of the tethers may extend out of plane (•) symbols of FIG. 3. In some embodiments, such as the embodiments represented by FIG. 2, needles may be positioned at a distal end of one or more tethers, allowing the tethers to be passed through the tissue wall without significant local damage (e.g., akin to a suture and needle). In other embodiments, the tethers may be passed through the tissue wall with the help of one or more surgical tools.

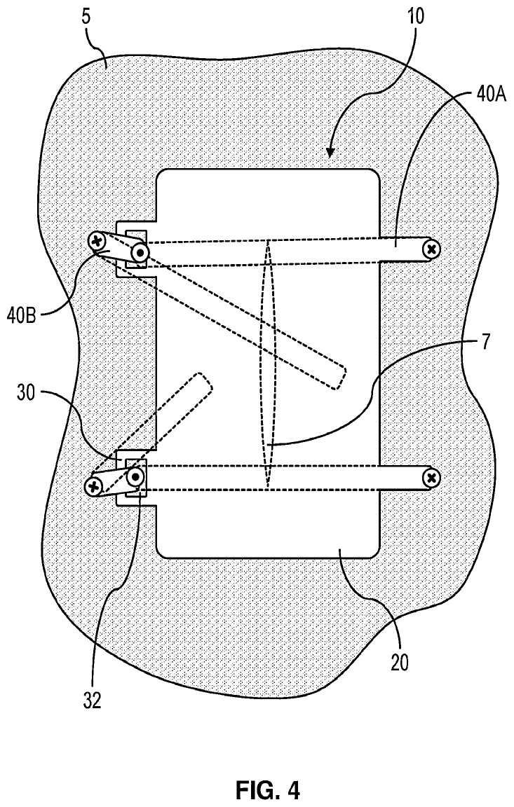

As shown in dashed lines in FIG. 3, an intermediate portion 40C of the tether may then span across the defect 7 (and/or any other suitable portion of the tissue wall 5), towards an eyelet 32. It should be appreciated that although FIG. 3 depicts an eyelet 32 enclosed by a tab 30, in some embodiments, the eyelet 32 may be located directly in the body. The distal portion 40B of the tethers may then be looped around the tabs 30 and secured at any suitable location (e.g., the body 20, eyelets 32, tissue 5, etc.), represented by the fixation (●) symbols of FIG. 4. The distal portion 40B of the tethers may be fixed to the tissue with any suitable fastener, including, but not limited to, sutures, tacks, staples, barbs, hooks, and/or any other suitable fastener. It should be appreciated that any other portion of the prosthesis 10, such as the main body 20, may also be fixed to the tissue wall 5 through any suitable fastener.

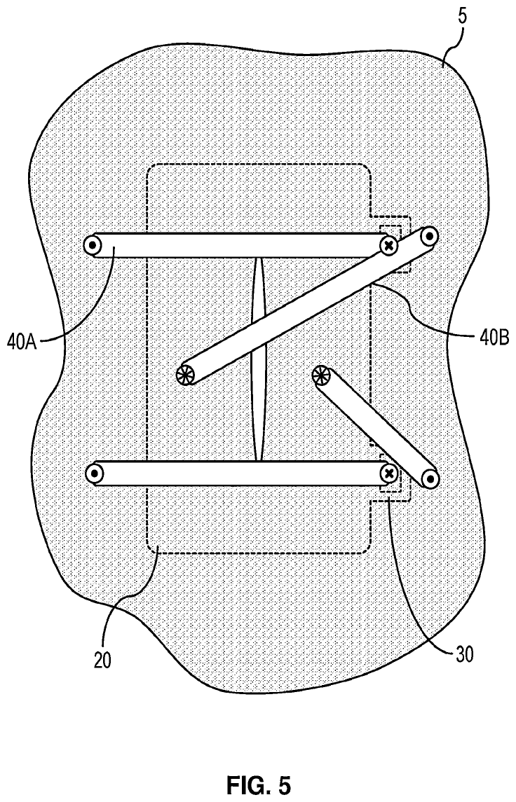

FIGS. 4-5 depict an implantable prosthesis 10 secured to a tissue or muscle wall 5 according to other embodiments. FIG. 4 shows an internal view of an implantable prosthesis 10 including tabs 30, eyelets 32, and tethers 40, whereas FIG. 5 shows an external view of the prosthesis secured to the tissue wall 5. The prosthesis 10 of FIGS. 4-5 may operate similarly to the prosthesis 10 of FIG. 3, but that after looping through the tabs 30, the distal portion 40B of the tethers may be passed back across the defect 7 and/or a portion of the tissue wall, as depicted by the dashed lines. The distal portion 40B may then be fixed at any suitable location. For example, the distal portion 40B may be fixed to the body 20 (as shown in FIG. 5), a portion of the tethers themselves, and/or the tissue wall 5. The tethers may be secured in any suitable manner, as the present disclosure is not limited by the means or manner with which the prosthesis is secured to the anatomical site.

It should be appreciated that the number of incisions (represented by the in-plane (x) and out of plane (•) symbols of FIGS. 3-5) in any embodiment may be adjusted by the operator. Thus, although three incision points are shown for a given tether in FIGS. 4-5, in some embodiments, the operator may choose to pierce the tether through an existing incision, thereby reducing the total number of incisions. In other embodiments, the operator may choose to form more than three incisions per tether. The operator may choose to make any suitable number of incisions to position and/or secure the prosthesis on the anatomical defect, as the present disclosure is not limited by the number of incisions made by the operator.

In some embodiments, passing a portion of the tethers across a defect, through an eyelet, and back across the defect may serve to distribute the forces applied by the tissue wall (e.g., intra-abdominal forces). In this way, the overall tension within one or more tethers and/or the tension within the fasteners used to fix one or more portions of the prosthesis to the tissue wall may be reduced. Accordingly, the risk of failure of the fasteners with which the tethers are secured may be reduced, as well as the risk of complications and relapse.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

What is claimed is:

1. An implantable prosthesis comprising:
a body of biologically compatible repair fabric, the body having a periphery;
at least one tether extending from a first portion of the periphery of the body; and
at least one eyelet associated with a second portion of the periphery of the body, the at least one eyelet configured to receive the at least one tether therethrough;
wherein the at least one tether is configured to span across a portion of the body to pass through the at least one eyelet.

2. The implantable prosthesis of claim 1, further comprising at least one tab extending from the second portion of the periphery of the body, wherein the at least one eyelet is located at least partially in the at least one tab.

3. The implantable prosthesis of claim 1, wherein the at least one eyelet is located on the body.

4. The implantable prosthesis of claim 1, wherein the at least one eyelet is located proximal to the second portion of the periphery of the body.

5. The implantable prosthesis of claim 1, wherein the at least one eyelet is located distal to the second portion of the periphery of the body.

6. The implantable prosthesis of claim 1, wherein the at least one eyelet is located within the periphery of the body.

7. The implantable prosthesis of claim 1, wherein the first portion of the periphery of the body is spaced apart from the second portion of the periphery of the body.

8. The implantable prosthesis of claim 1, wherein the first portion of the periphery of the body is located opposite from the second portion of the periphery of the body.

9. The implantable prosthesis of claim 1, wherein the at least one tether comprises a plurality of tethers, wherein the at least one eyelet comprises a plurality of eyelets, and wherein each of the plurality of tethers corresponds to at least one of the plurality of eyelets.

10. The implantable prosthesis of claim 1, wherein the body is formed of a mesh material.

11. The implantable prosthesis of claim 10, wherein the mesh material is configured to allow tissue ingrowth.

12. The implantable prosthesis of claim 1, wherein the body is formed of a resorbable material.

13. The implantable prosthesis of claim 1, wherein the body is formed of a non-resorbable material.

14. The implantable prosthesis of claim 1, wherein the body is configured to cover an anatomical defect.

15. The implantable prosthesis of claim 1, wherein the body is configured to be used in a ventral hernia repair procedure.

16. The implantable prosthesis of claim 1, wherein a width of the at least one tether is at least 5% of a length of the body.

17. The implantable prosthesis of claim 1, wherein a length of the at least one tether is at least 120% of a width of the body.

18. The implantable prosthesis of claim 1, further comprising a needle located at a distal end of the at least one tether.

19. The implantable prosthesis of claim 1, wherein the at least one tether is configured to exhibit a first visual appearance in an untensioned configuration, and wherein the at least one tether is configured to exhibit a second visual appearance in a tensioned configuration.

20. A method of repairing a tissue defect, the method comprising acts of:
(a) delivering an implantable prosthesis to a tissue defect site on a tissue wall, the implantable prosthesis comprising:
a body having a periphery,
at least one tether extending from a first portion of the periphery, and
at least one eyelet associated with a second portion of the periphery;
(b) positioning the body over the tissue defect on a first side of the tissue wall;
(c) extending the at least one tether across a second side of the tissue wall located opposite the first side of the tissue wall; and
(d) inserting the at least one tether through the at least one eyelet on the first side of the tissue wall to secure the body over the tissue defect.

21. The method of claim 20, further comprising prior to act (c), passing the at least one tether through a first portion of the tissue wall adjacent to the tissue defect from the first side to the second side of the tissue wall.

22. The method of claim 20, further comprising following act (c), passing the at least one tether through the second portion of the tissue wall from the second side to the first side thereof.

23. The method of claim 20, wherein act (d) further comprises adjusting the at least one tether with the at least one eyelet to secure the body over the tissue defect.

24. The method of claim 20, further comprising securing the at least one tether to the second side of the tissue wall.

25. The method of claim 20, wherein act (c) further comprises passing the at least one tether through at least one tab associated with the second portion of the periphery of the body, wherein the at least one eyelet is located at least partially in the at least one tab.

26. The method of claim 20, wherein the at least one tether comprises a plurality of tethers and the at least one eyelet comprises a plurality of eyelets, and wherein acts (c) and (d) are performed for each of the plurality of tethers.

27. The method of claim 20, wherein the implantable prosthesis is formed of a mesh material.

28. The method of claim 20, wherein the implantable prosthesis is configured to allow tissue ingrowth.

29. The method of claim 20, wherein the implantable prosthesis is formed of a resorbable material.

30. The method of claim 20, wherein the implantable prosthesis is formed of a non-resorbable material.

31. The method of claim 20, wherein a width of the at least one tether may be at least 5% of a length of the body.

32. The method of claim 20, wherein a length of the at least one tether may be at least 120% of a width of the body.

33. The method of claim 20, wherein act (c) further comprises passing a needle through the first portion of the tissue defect, the needle located at a distal end of the at least one tether.

34. The method of claim 20, further comprising an act (e) tightening the at least one tether with the at least one eyelet to secure the body over the tissue defect.

35. The method of claim 34, wherein act (e) further comprises changing a visual appearance of the at least one tether after tightening the at least one tether.
